(19) United States
(12) Patent Application Publication
(10) Pub. No.: US 2007/0224597 A1
(43) Pub. Date: Sep. 27, 2007

(54) ISOLATING FETAL TROPHOBLASTS

(75) Inventors: Tony Pircher, San Diego, CA (US); Roberto Fagnani, San Diego, CA (US)

Correspondence Address: COOLEY GODWARD KRONISH LLP;ATTN: Patent Group, Suite 500, 1200 - 19th Street, NW, WASHINGTON, DC, 20036-2402, US

(73) Assignee: BIOCEPT, INC., San Diego, CA (US)

(21) Appl. No.: 11/277,288

(22) Filed: Mar. 23, 2006

(51) Int. Cl. C12Q 1/68; C12N 5/06

(52) U.S. Cl. 435/6; 435/455; 435/358

(57) ABSTRACT

Methods for isolating and purifying fetal trophoblasts from a mucus sample obtained from the uterine cavity of a pregnant female. The mucus sample is transported from a clinical collection facility to a laboratory in a transportation medium so the cells remain viable. The mucus sample is then subjected to precise processing steps, including treatment with mucolytic agents or mucinases, sugar hydrolysis enzymes, nucleases, and proteases to provide fetal cells, the outer surfaces of which are so essentially completely devoid of attached mucosal biological material that they are then isolated in greater numbers than previously had been possible. The isolated cells are in appropriate condition to immediately be effectively subjected to FISH or to other molecular diagnostics.

### FIELD OF THE INVENTION

This invention relates to methods for the isolation of fetal trophoblast (placental) cells obtained from a pregnant female mammal and more particularly to treatment of a cervical mucus sample with reagents useful to liberate trophoblasts, and still more particularly to methods for providing a sample of fetal trophoblasts acceptable for testing by FISH or the like within about 8 hours after a sample obtained from a pregnant female is received in a laboratory facility.

### BACKGROUND OF THE INVENTION

Cells derived from the fetus enable genetic and/or biochemical information about the fetus to be obtained. By isolating trophoblast cells early in pregnancy, these cells may be used to obtain fetal genetic and/or biochemical information and particularly to detect human fetal abnormalities.

Prenatal testing has been carried out for many years on fetal cells obtained by either amniocentesis or chorionic villous sampling (CVS). Amniocentesis may normally be performed at about 16 weeks of gestation and requires skilled personnel to insert a needle into the amniotic sac of the fetus and remove between 20-30 ml of amniotic fluid. The amniotic fluid contains fetal cells upon which subsequent tests may then be performed. There is however a risk of inducing a spontaneous abortion associated with this method of obtaining fetal cells. Moreover, if genetic diagnosis of the fetal cells following this 16-week term procedure reveals an abnormality, the prospect of a mid-trimester pregnancy termination can be both psychologically stressful and associated with some risk to the mother.

Chorionic villous sampling also requires the involvement of skilled personnel to take a small biopsy from the placenta of an 8-12 week old fetus, and it likewise has a risk of inducing a spontaneous abortion. However, earlier diagnosis of any chromosomal abnormality may make CVS more attractive than amniocentesis.

The need for skilled personnel and the possibility of inducing spontaneous abortion for both these procedures has generally meant that such prenatal genetic assessments are made only on pregnant women who are deemed to have a fairly high risk of carrying a fetus with a chromosomal abnormality. Attempts to provide simpler procedures have involved obtaining blood from an arm vein or from the uterine wall of a pregnant female, and extracting fetal cells which are normally sloughed off from the placenta and are now generally agreed to be present in the maternal bloodstream. Such non-invasive isolation of fetal cells negates any risk of inducing a spontaneous abortion.

U.S. Pat. No. 5,503,981 provides a method for the isolation of trophoblast cells from a blood sample of a pregnant mammal by contacting the blood sample with an effective amount of an antibody specific for villous syncytiotrophoblast and non-villous cytotrophoblast cells. Cells bound by this antibody are separated from the sample, and the isolated cells are used to obtain genetic and/or biochemical information.

Although the identification and isolation of fetal cells from a maternal blood sample would seemingly provide a desirable, non-invasive alternative method for acquiring fetal genetic material for prenatal genetic testing, in practice a major drawback lies in the extreme rarity of fetal cells in maternal blood. It has been determined that trophoblast cells are only present in very small concentrations in the maternal bloodstream; thus, procedures for separation from maternal blood have proved to be problematic and timestaking. Although advances have made several improved detection methods available, including polymerase chain reaction (PCR) and fluorescence in situ hybridization (FISH), a major difficulty still persists in the routine use of maternal blood for prenatal diagnosis; it is the inability to reasonably enrich and/or isolate the very small number of fetal cells present in mixture with maternal cells in order to yield truly reliable diagnostic results. Such isolation is a necessity because there is little tolerance for maternal DNA-containing cells in many diagnoses; for example, in molecular diagnosis, substantially zero tolerance is generally permitted.

As a consequence, this extreme rarity of fetal cells in maternal blood has resulted in a number of specialized techniques having been designed to attempt to enrich and/or isolate the fetal cell fraction or the fetal genetic material from maternal blood. U.S. Pat. No. 5,432,054 discloses an enrichment method that employs gradient centrifugations for isolating fetal cells. Typically such a method has not been sensitive enough to effect the isolation of a fetal cell fraction usable for highly reliable genetic testing, e.g., substantially zero tolerance.

A labeled antibodies approach, disclosed in U.S. Pat. No. 4,675,286, has also been utilized in an attempt to isolate fetal cells from a maternal blood sample by employing flow cytometry to effect separation of these cells from maternal cellular components. However, limitations inherent in flow cytometry sorting have also prevented such methods from being widely practiced for this purpose. A major limitation inherent to such flow cytometry techniques arises from the antibodies utilized by such techniques. Such antibodies, although generated to be cell-specific, often crossreact with other unwanted cell types which are present in far higher concentration in the sample. As a result, although such methods may be sufficient to enrich the mixture in fetal cell types, they often cannot be used for reliable, zero tolerance, fetal cell isolation.

U.S. Pat. No. 5,580,724 discloses a method for obtaining cells of fetal origin from a maternal blood sample by using a centrifugation process to first isolate mononuclear cells (MNC). After removing the plasma and medium, the layer of MNC is washed and cultured for seven days in a specific medium that contains stem cell factor (SCF), erythropoietin and II-3 and II-6 in a high humidity atmosphere containing 5% carbon dioxide. Non-adherent cells are recovered by aspirating, and cells are then replated and cultured for 14 days under conditions conducive to fetal stem cell growth. After 21 days the cells are counted, plated and examined. The long time delay and expense has prevented its adoption as a clinical practice.

U.S. Pat. No. 6,221,596 teaches a method for isolating a rare cell type, such as trophoblasts, from a sample of maternal blood, which includes a mixed population of cells by first providing a magnified image of a portion of the sample. Rare cell types within the population of cells are morphologically identified, and the identified rare cell types are retrieved using a micromanipulator. This method requires skill and special instrumentation, and it is timestaking.

Because of these, at least perceived, shortcomings, other options have been explored, with particular attention being given to obtaining cells that are present in the uterine cavity. U.S. Pat. No. 4,675,286 teaches obtaining samples of detached cells from the cervical cavity, which samples will include fetal cells originating from the placenta mixed with maternal cells originating from the cervical endometrium and the placenta. Such cells are obtained from the uterine cavity through the uterine canal by a swab or other collecting tool which is inserted through the mucus plug of the uterine canal. It is then attempted to separate the fetal cells from the maternal cells in the mixture by treating the cell mixture with microspheres that carry antibodies specific to fetal trophoblasts. Fetal cells captured on the microspheres are then propagated in a culture medium and later removed from the microspheres by agitation for examination. This general procedure, which was disclosed at least as early as 1987, has not achieved widespread use, and improvements upon it have been sought.

PCT application WO 2004/087863 proposes to diagnose for gender and potential chromosomal abnormalities by obtaining transcervical cells from a pregnant female, as by using a Pap smear cytobrush and shaking the brush into a test tube containing a few milliliters of a tissue culture medium that contains a penicillin/streptomycin antibiotic. The sample is then subjected to cytocentrifugation, and the resultant cytospin slides are kept in 95% alcohol until subjected to immunological staining, using an antibody directed against a trophoblast antigen, with numerous such antibodies being described. This staining is then followed by counterstaining the cells, as by dipping the slides in an appropriate solution, and the trophoblast cells are marked. Once the desired cells are marked, the staining may be removed, and FISH analysis is carried out using a two color technique and directly-labeled probes. FISH signals from such cells can be viewed using a fluorescent microscope. This course of action analyzes fetal trophoblasts essentially individually, while they remain a part of a plated mixture of fetal and maternal cells. It requires much sophisticated equipment and highly trained operators, and for such reason, it has not been favored.

Published U.S. Application 2005/0123914 also recognizes that obtaining a cervical mucus sample provides a prospective basis for noninvasive, prenatal diagnosis of fetal chromosomal abnormalities. It describes first obtaining a cervical mucus sample during the first trimester of pregnancy, as by using a transcervical swab. The mucus is then treated with a mucolytic agent followed by treatment with a collagenase and a protease, and it is indicated that commercially available mixes of enzymes are used to dissociate the cells from the mucus material. The cells are retrieved by washing, followed by centrifugation to separate them from the supernatant. Treatment with fetal specific antibodies is used to isolate fetal cells from the mix of fetal and maternal cells remaining after washing. It is proposed to identify the fetal cells by treating with a cocktail of three antibodies, namely, NDOG1, NDOG5 and FT1.41.1, which antibodies are fluorescently labeled. The fetal cells are then separated using fluorescent activated cell sorting (FACS), magnetic bead separation, micromanipulation and/or laser capture and fluorimmunihistochemistry; micromanipulation is said to be preferred. Once the fetal cells are obtained, there are a number of processes which are described that can be used for diagnosis. Although the overall procedures described therein basically provide an attractive path for prenatal diagnosis of potential genetic disorders, room for improvement in various of the steps remains.

As a result improved methods for providing a sample of isolated trophoblast cells, and particularly mononucleated trophoblasts as opposed to multinucleated trophoblasts (because it has been shown that mononucleated trophoblasts generate more reliable and consistent data when subjected to chromosomal analysis, such as FISH), have continued to be sought after.

### SUMMARY OF THE INVENTION

The invention provides methods for isolating and purifying fetal trophoblasts in a sample containing trophoblasts and maternal cells obtained from a pregnant female. A mucus sample obtained from the uterine cavity is added to a selective maintenance medium in a transportation tube and maintained at a temperature of between about 4° C. and 20° C.; the medium may optionally be treated so that atmosphere within the tube contains not greater than about 4% oxygen. The character of the medium is such that the trophoblasts in the mucus sample are maintained in a viable state, thus allowing transportation from a clinical collection facility to a laboratory equipped for analysis. Following transportation, the mucus sample is subjected to precise processing steps, including treatment with enzymes, such as mucolytic agents or mucinases, sugar hydrolysis enzymes, nucleases, and proteases. The result is a product of fetal and maternal cells, the outer surfaces of which are so essentially completely devoid of attached mucosal biological material thereof that isolation of fetal cells in greater numbers than previously had been obtained from such a sample is possible, and which cells are essentially totally devoid of maternal cells and can immediately be effectively subjected to FISH or to other molecular diagnostics.

In one particular aspect, the invention provides a method for quickly and accurately obtaining chromosomal analysis of fetal trophoblast cells from a sample obtained from a pregnant female mammal which contains such cells and others, which method comprises the steps of (a) obtaining a sample of cervical mucus from a pregnant female mammal that contains fetal trophoblast cells and maternal cells, which sample was collected on a collection implement and deposited in a selective preservation medium that is favorable to the preservation of trophoblasts as opposed to maternal cells; (b) removing said implement from said preservation medium and treating said sample and collection implement with a combination of a mucolytic agent and with a sugar hydrolysis enzyme and incubating at 35 to 40° C., (c) treating said sample with a combination of a nuclease and a protease and incubating at 35 to 40° C., (d) removing said collection implement, optionally adding EDTA or a detachment enzyme, and centrifuging to concentrate cells and other biological material from said sample, (e) removing supernatant following said centrifuging; (f) adding nutrient medium suitable to culture CHO cells and mixing, (g) centrifuging to again concentrate said cells and other biological material and removing supernatant, (h) causing a suspension of said product of step (g) in an aqueous buffer containing sodium azide, to flow through a microchannel device having a collection region wherein surfaces are coated with sequestering agents that are specific to trophoblast cells and not found on maternal cells so as to effectively capture same to the substantial exclusion of maternal cells, and (i) identifying said captured trophoblast cells and analyzing said identified cells.

In another particular aspect, the invention provides a method for quickly and accurately obtaining chromosomal analysis of fetal trophoblast cells from a sample obtained from a pregnant female mammal containing such cells and others, which method comprises the steps of (a) obtaining a sample of cervical mucus from a pregnant female mammal that contains fetal trophoblast cells and maternal cells, which sample was collected on a collection implement; (b) treating said sample with a combination of a mucolytic agent and a sugar hydrolysis enzyme and incubating at 35 to 40° C., (c) treating said sample with a combination of a nuclease and a protease and incubating at 35 to 40° C., (d) centrifuging to concentrate cells and other biological material from said sample, (e) resuspending said cells in an aqueous buffer which optionally includes a stabilizing agent, and (f) separating said trophoblasts from said maternal cells by the use of sequestering agents which are specific for antigens on the outer surfaces of said trophoblasts.

In a further particular aspect, the invention provides a method for quickly and accurately obtaining a chromosomal analysis of fetal trophoblast cells from a sample of cervical mucus from a pregnant female mammal, which method comprises the steps of (a) obtaining a sample of cervical mucus on a collection implement from a pregnant female mammal, which sample contains fetal trophoblast cells and maternal cells; (b) adding said collection implement containing said mucus to a transportation medium of such a character that said trophoblast cells are maintained in a healthy state while some maternal cells expire, whereby the percentage of fetal trophoblast cells therein increases, (c) removing said collection implement carrying said mucus from said transportation medium and treating said collection implement and said mucus with mucolytic agents, a sugar hydrolysis enzyme, nucleases and proteases in a tube and incubating at a temperature between 35 to 40° C. so as to cause extraneous biological components of said mucus to be detached from the outer surfaces of the trophoblast cells, (d) removing said collection implement from said tube following said incubating and depositing said implement in a second tube (e), treating said collection implement and said remaining mucus with mucolytic agents, a sugar hydrolysis enzyme, nucleases and proteases in said second tube and incubating at a temperature between 35 to 40° C., removing said treatment media from both said first and second tubes and resuspending said cells from said sample in a culture media suitable to grow CHO cells to wash said cells and remove extraneous biological material derived from said mucus, (e) resuspending said cells in an aqueous buffer containing a stabilizing agent and sodium azide to provide a liquid suitable for flow through a microflow separation device, (f) separating said trophoblast cells from said remaining maternal cells in said microflow device through the use of sequestering agents which bind to antigens on the outer surfaces and trophoblast cells, and (g) then carrying out chromosomal analysis upon said separated trophoblast cells, whereby said analysis is completed within 8 hours of when said collection implement carrying said mucus is removed from said transportation medium.

### DETAILED DESCRIPTION OF THE PREFERRED EMBODIMENTS

Basically, a cervical mucus sample is collected from a pregnant female mammal and trophoblast (placental) cells are isolated therefrom. Described hereinafter are the steps employed and the media and reagents useful for performing these steps. Certain preferred methods of obtaining fetal cells from a cervical mucus sample from a pregnant female mammal are specifically described along with the effective isolation of fetal trophoblast cells from this cervical mucus sample, which results in trophoblast cells in a condition so that they can be analyzed by FISH or other molecular diagnosis.

Samples of cervical mucus containing fetal and maternal cells are often obtained by clinicians at various locations, who must then transport them to a laboratory for processing, and such samples are preferably preserved in an aqueous preservation medium in a capped vial or tube, as by depositing the appropriate portion of a cervical mucus collection device (e.g. cytobrush, cytobroom, or swab) therein. Trophoblast cells should not be frozen during transportation in order to keep the cells most viable for later processing; instead, they are preferably maintained at about 4° C. and not higher than about 20° C.

Because trophoblast cells are at least partially embedded in cervical mucus, which is composed of protein-polysaccharide complexes called mucopolysaccharides (also glycosaminoglycans) and other macromolecules, cervical mucus samples which have been obtained on a brush or the like need to be carefully treated to remove all of such mucosal biological material from the exterior cell surfaces.

The treatment of a mucus sample that is described hereinafter effectively eliminates essentially all non-cellular components while also selecting for viable trophoblasts. The use of a trophoblast-enriching initial transportation medium and then other media during subsequent processing has been found to be useful in preserving delicate trophoblast cells. At the same time, treatment methods described hereinafter discriminately eliminate non-trophoblast components while concurrently separating tenacious biological material from the surfaces of fragile trophoblast cells.

The following is a description of a generalized treatment procedure for purifying trophoblasts from a cervical mucus sample upon arrival at an analytical laboratory. It is illustrative of such a process for treating mucus-bound cells, but it is in no way intended to be limiting.

A capped collection tube containing a cervical mucus sample, a collection device, and transport media arrives at a laboratory and is logged into the laboratory's data management system. A representative transport media is used which comprises:

Low Calcium Basal Medium

| Component | g/L |
|---|---|
| Inorganic Salts | |
| Soluble Calcium | 0.005 |
| Soluble Magnesium | 0.1 |
| Potassium Chloride | 0.1 |
| Sodium Bicarbonate | 1.0 |
| Sodium Chloride | 7.5 |
| Sodium Phosphate Dibasic (anhydrous) | 0.3 |
| Minor amount of other minerals | |
| Amino Acids | |
| L-Alanine | 0.01 |
| L-Arginine (free base) | 0.2 |
| L-Asparagine (anhydrous) | 0.02 |
| L-Aspartic Acid | 0.01 |
| L-Cystine-2HCl | 0.04–0.06 |
| L-Glutamic Acid | 0.02 |
| L-Glutamine | 0.3–0.9 |
| Glycine | 0.01 |
| L-Histidine (free base) | 0.015 |
| L-Isoleucine | 0.002 |
| L-Leucine | 0.06 |
| L-Lysine HCl | 0.02 |
| L-Methionine | 0.005 |
| L-Pnenylalanine | 0.005 |
| L-Proline | 0.03 |
| L-Serine | 0.06 |
| L-Threonine | 0.01 |
| L-Tryptophan | 0.003 |
| L-Tyrosoine 2 Na-2H₂ | 0.0035 |
| L-Valine | 0.035 |
| Vitamins | |
| D-Biotin | 0.0002 |
| Choline Chloride | 0.003 |
| Folic Acid | 0.001 |
| myo-Inositol | 0.035 |
| Niacinamide | 0.001 |
| p-Amino Benzoic Acid | 0.001 |
| D-Pantothenic Acid (hemicalcium) | 0.00025 |
| Pyridoxine-HCl | 0.001 |
| Riboflavin | 0.0002 |
| Thiamine-HCl | 0.0001 |
| Vitamin B-12 | 0.000005 |
| Other | |
| D-Glucose | 1.0–2.0 |
| Glutathione (reduced) | 0.001 |
| Phenol Red-Na | 0.0053 |
| pH at RT (with sodium bicarbonate) | 7.3 ± 0.3 |

Following data entry and requisite paperwork, the tube is forwarded to laboratory technicians who follow safety guidelines, including the use of a biohazard hood, when such human biological materials are being handled in the treatment process described hereinafter. The collection brush is removed from the transportation medium and placed in a 15 ml tube, such a 15 ml tube that contains a culture media of the type that has been formulated to grow Chinese hamster ovary (CHO) cells, such as Hams F-12 media. This tube is hereinafter referred to as a sample treatment tube.

Initially, the tube containing the collection brush and mucus sample is incubated in a 37° C. water bath for 30 minutes to bring it to such temperature before being treated with certain chemical reagents. It has been found that initial treatment should be with a combination of a mucolytic agent or mucinase and a sugar hydrolysis enzyme. Such initial treatment in the sample tube with a mucinase and a sugar hydrolysis enzyme effectively liberates trophoblasts from some components of mucus and sugar residues.

N-acetyl-L-cysteine is a mucolytic agent that is useful in reducing the viscosity of the cervical mucus; it thus aids in the physical release of mucin from the collection device into the media in the treatment tube where it can be processed. N-acetyl-L-cysteine liquefies mucus by breaking down mucopolysaccharides (also glycosaminoglycans) into smaller molecular subunits. Although N-acetyl-L-cysteine is the preferred mucolytic agent, other known mucinases that may alternatively be used to hydrolyze cervical mucus, including dithiothreitol (DTT), bromhexine hydrochloride, L-cysteine, and the hyaluronidases, such as hyaluronate lyase, hyaluronoglucosaminidase, and hyaluronglucuronidase.

β-galactosidase is the preferred sugar hydrolysis enzyme; it is useful for cleaving carbohydrate chains which surround trophoblast cells in the mucus. β-galactosidase hydrolyzes the β-galactosidase linkage between glucose and galactose, releasing the cells from the glycoproteins in the mucus. There are other sugar hydrolysis enzymes that may alternatively be used to hydrolyze sugar residues, for example, invertase.

Following the addition of at least one mucinase and at least one sugar hydrolysis enzyme to the sample treatment tube, an aqueous solution of calcium chloride is added, and the sample is incubated for a suitable period of time, i.e. at least about 10 minutes, and preferably about 30 minutes, at a temperature of 35 to 40° C., and preferably at 37° C. on a tube rocker so as to activate the enzymes. After this incubation with the mucolytic agent and the sugar hydrolysis enzyme, a combination of enzymes is added to the tube, followed by incubation on a tube rocker for a few minutes; the enzyme mixture that is used comprises a nuclease, a protease and preferably an additional amount of a sugar hydrolysis enzyme, preferably β-galactosidase.

Nucleases serve to enzymatically cleave extracellular single and/or double stranded DNA and RNA. The use of nucleases to hydrolyze extracellular DNA has been shown to degrade mucosal secretions as reported by Duplantier et al. in US Pharmacist, 17:34-52 (1992). Endonucleases are preferred over exonucleases because they cleave DNA at several interior positions, whereas exonucleases only digest nucleotides from the end of a DNA strand. Thus, endonucleases should provide a more thorough digestion in comparison to exonucleases. However, under certain circumstances exonucleases may be used instead of, or in addition to, endonucleases. Restriction enzymes, which are endonucleases that cleave in specific regions, may also be utilized; non-specific endonucleases or nickases are preferred. Preferably, a DNase which degrades single stranded and double stranded DNA is used, and more preferably DNase I, a nickase, is added to the sample treatment tube to digest extracellular DNA. Examples of other nickases that may be used include Mung bean nuclease (digests single stranded DNA and RNA) and Benzonase® (degrades DNA and RNA in many forms to small oligonucleotides and promotes quick reduction of cell lysate viscosity, which is useful for ultracentrifugation). Eurogentec USA (San Diego, Calif.) offers each of the aforementioned endonucleases. Because mucus samples derived from the cervix contain a heterogeneous collection of biological material including intact cells (both maternal and fetal), lysate from lytic cells, extracellular proteins, and extracellular DNA, nucleases are considered most useful for degrading exogenous DNA which, if not eliminated, may negatively affect test results and/or cause difficulty in sample processing. Degradation of extraneous DNA further frees the cells of interest, i.e. trophoblasts, from surface contaminations.

Proteases (or proteinases) hydrolyze the protein portions of the mucus. Because cervical mucosal samples contain extracellular proteins, performing proteolytic cleavage on extracellular proteins yields a cleaner sample by further isolating trophoblast cells from their original heterogeneous environment and freeing the surface antigens so they can assume their native 3-D configurations. In one example, an enzyme cocktail, such as pronase which cleaves almost any peptide bond, is used to digest extracellular proteins in a sample tube. Pronase includes both endo-proteinases and exo-proteinases. Numerous proteolytic compounds that are useful for hydrolyzing proteins are known in the art. Many of these compounds, such as trypsin, chymotrypsin, pepsin, and papain, may be used in addition to or in lieu of pronase.

Simultaneous treatment using this combination of a nuclease, preferably DNase I, a protease, preferably Pronase, and β-galactosidase has been found to be extremely effective in totally liberating trophoblast cells from the tenacious attached biological material of cervical mucus. In addition to the above-described mucolytic agents, sugar hydrolysis enzymes, nucleases and proteases that are used in the processing of the mucus samples, other supplemental chemical reagents may also be included, but such are not considered necessary. However, if included, it should be recognized that certain enzymes may require incubation at a slightly higher temperature to activate, and some enzymes, if left unchecked, may have a tendency to over-degrade certain biological components. Therefore, it may be desirable to halt digestion by some enzymes by quenching, e.g. either by exposure to a second reagent or by effecting a significant drop in the reaction temperature. In the preferred procedure, the collection brush is removed from the first treatment tube after about two minutes, and it is placed in a labeled second treatment tube. However, incubation of the first tube is continued for about 10 more minutes after such removal.

The second sample treatment tube, in which the collection brush was deposited, contains a similar amount of Hams F-12 media, a mucolytic agent, a sugar hydrolysis enzyme and preferably calcium chloride; the tube and contents have been prewarmed in a 37° C. water bath for at least ten minutes. Treatment of the collection brush in the second tube is then carried out similar to that just described. After treatment with the enzyme mixture and incubation for about 3 minutes, the collection brush is removed from the second sample treatment tube and placed in a third sample treatment tube (having similar contents to the second treatment tube), and the preceding treatment is repeated. Following incubation in the third sample treatment tube for about 5 minutes, the collection brush is withdrawn, stored and labeled. Once the collection brush has been removed from each of the respective three treatment tubes and the further incubation is completed, the contents of each tube are treated in the same manner as set forth hereinafter. During this entire treatment process, it has been found that far superior results are obtained if a temperature in the range of 35° to 40° C. is continuously maintained.

Following the completion of the incubation after removal of the collection brush, EDTA or EGTA and/or a detachment enzyme, for example, Accumax A7089, is added to the tube. Although a suitable detachment enzyme may be utilized, in the preferred embodiment, EDTA is used. After mixing with the cell suspension, the tube containing the EDTA is centrifuged at about 37° C. for about 5 minutes. In each of the three sample tubes, after the addition of EDTA and centrifuging, the media is vacuum-aspirated, leaving about ½ ml of media in the tube with the pellet. At this point, the cells have been very effectively liberated from the tenacious mucosal components initially adhering to the cell surfaces. The cell pellet is then resuspended in Hams F-12 media two separate times and centrifuged as washing operations. Following this second washing, the pellet is resuspended in 5 ml of a buffer solution of a character that will be used in a subsequent cell separation step using a microflow device that employs trophoblast-selective antibodies.

Such washing separates trophoblast cells from digested or partially digested biomolecules, and subsequent centrifugation results in a separation between the cells located at the bottom of the tube and the supernatant, which includes the media and small molecular weight biomolecules. Although the term trophoblast “cell” is used throughout this application, it should be understood to include cell fragments and/or remnants that would likewise carry the surface ligands specific to the sequestering agents. Following centrifugation, and taking care not to disturb the pellet, vacuum aspiration removes a large portion of the supernatant (e.g. 80 percent or more), and the remaining pellet is then resuspended for the next step As mentioned above, all these steps are best performed at the desired temperature of about 37° C.

In each of the three sample tubes, after the addition of EDTA, centrifuging and vacuum-aspirating to remove most of the media as described above, the cell pellet is resuspended in Hams F-12 media two separate times, and centrifuged as a further washing operation. Following this second washing, the pellet is resuspended in 5 ml of a buffer solution of a character that will be used in a subsequent cell separation step using a microflow device that employs trophoblast-selective antibodies.

The composition of the separation buffer which is used may vary according to the character of the next step for processing the sample to selectively separate the trophoblasts. Examples of reagents that the separation buffer may include are tissue culture media, enzymes, and stabilizing agents. For example, bovine serum albumin (BSA) may be used as the stabilizing agent. Alternatively, fetal bovine serum (FBS), bovine serum, calf serum, newborn calf serum, goat serum, horse serum, human serum, chicken serum, porcine serum, sheep serum, embryonic bovine fluid, rabbit serum, and the like may be used (all of the foregoing reagents are available through Proliant Biologicals). The trophoblast cells collected from the initial tube and from the second and third tubes may be combined and resuspended in a total of about 1 ml of buffer. It has been found that the inclusion of a small amount of sodium azide in the aqueous buffer in which the cells are suspended, prior to the ultimate separation step, provides improved results. By including from about 0.05 to about 0.2% by weight of sodium azide, it is found that any tendency which antigens normally on the surface of the trophoblast cells might have to internalize is overcome; thus, these antigens which are indicative of fetal trophoblast cells remain prominent where they can attach to sequestering agents.

The trophoblasts, which have now been freed from other biological components of the mucus, are preferably isolated from the remaining maternal cells in a microchannel device, such as that disclosed in pending U.S. patent application Ser. No. 11/038,920, filed Jan. 18, 2005. The interior of the microchannel device includes a collection region with a pattern of transverse posts. Surfaces throughout the region are derivatized and are preferably provided with a coating that facilitates the direct or indirect attachment of sequestering agents specific to the targeted biomolecules of interest.

The term sequestering agent refers to a material capable of interacting in a specific fashion with the target cell to physically sequester the cell. The preferred sequestering agents for trophoblasts are immunoglobulins (particularly antibodies) directed against antigens on the trophoblast surface. However, complex carbohydrates or synthetic molecules may alternatively be used.

Attachment of the sequestering agents, such as antibodies (Abs), throughout the collection region is effected in a manner so that the sequestering agents perform efficiently; this is accomplished by preferably coating the separation surfaces with a thin layer (at least about 1 μm thick) of a particular hydrophilic hydrogel substance which is an isocyanate-functional polymer containing PEG, PPG or a copolymer thereof of a MW of about 3,000 to 6,000 daltons, that is polymerized by urethane bonds and that contains reactive isocyanate groups. Details of the formulation of such coating material are disclosed in a co-pending U.S. patent application Ser. No. 11/021,304, filed Dec. 23, 2004.

Sequestering agents can be directly or indirectly attached to the hydrogel coating; however, indirect immobilization may be preferred. Such contemplates the employment of an intermediate agent or substance that is directly linked to the coating; for example, one member of a coupling pair may be attached to the hydrogel coating as an intermediate agent. Streptavidin or an antibody (Ab) directed against an antibody of another species might be so attached; such intermediate would thereafter couple to a biotinylated Ab or to an Ab of such other species. For example, avidin may be included as a part of an aqueous polyurethane prepolymer composition used to effect the coating. Avidin thus becomes covalently linked to isocyanate groups in the coating, and it then facilitates attachment of desired biotinylated antibodies which are specific to trophoblast cells. The use of Abs as sequestering agents is preferred for trophoblast cell separation, and such antibodies may also be directly bound by incorporating the Abs in the coating material being applied. For example, the antibody in aqueous solution can be mixed with a polyurethane prepolymer having free isocyanate or equivalent groups, such as a polyether isocyanate, and as a result, the surfaces of the collection region will become coated with a layer of such Abs. Particularly preferred is the use of a prepolymer having free isocyanate groups which provides a hydrophilic, polyurethane-based, hydrogel layer upon polymerization.

Instead of using a hydrogel layer on the separation surfaces, Abs may, for example, be first treated with 2-aminothiolane to thiolate them, and the resulting thiolated Abs may then be conjugated with posts that have been treated with PEG-maleimide. Alternatively, the desired Abs may be directly covalently bonded to an appropriate hydrophilic coating on the posts having reactive isocyanate groups or thiocyanate groups.

With the antibodies attached throughout the patterned post collection region of the microchannel device, the buffer suspension containing the target cell population is caused to flow through the collection region, as by being discharged carefully from a standard syringe pump into an inlet passageway or drawn therethrough by a vacuum pump or the like from a sample reservoir at the inlet.

Following the completion of the passage of the liquid sample through the device, the trophoblast cells will have been captured within the collection region. Washing is then carried out with buffers so as to remove non-specifically bound cells and any remaining biomaterial. Washing with effective buffers purges the region by removing such nonspecifically bound material and leaving only the desired target cells attached in the collection region.

Once washing with buffers has been completed, the collection region is preferably filled with a chemical reagent that will cause the captured cells to be suitably released. Release is effected by a suitable method as known in this art, such as chemically (e.g. change in pH) or through the use of enzymatic cleavage agents or the like. For example, a reagent may be applied to cleave a sequestering agent, or to cleave the bond between such agent and the cells, in order to release the target cells from linked or coupled attachment to the surfaces in the collection region. For example, if the cells have been sequestered through the use of antibodies that are specific to surface characteristics of the target cells, release may be effected by treating with a solution containing trypsin or another suitable protease, such as Proteinase K. Alternatively, a collagenase may be used to effect release from other sequestering agents, or a specifically cleavable linker may be used to attach the sequestering agent to the collection surfaces.

During such cleavage, the inlet and the outlet from the microchannel device are preferably plugged with simple stoppers, and the device is then subjected to centrifuging following such release. The centrifuging may be carried out at a speed equal to about 500 g for about 5 minutes with the stoppers in place and with the device oriented so that centrifugal force presses the targeted biomolecules against the surface of a glass slide that forms one surface of the collection region. At the completion of the centrifuging, substantially all of the targeted trophoblasts collected in the collection region are now adhering to the top surface of the slide. Disassembly of the device is then carefully effected to provide the slide with the trophoblasts disposed on its upper surface.

Should it be decided to subject the isolated trophoblasts to FISH analysis, the trophoblasts may first be treated with methanol. The cells adhering to the surface of the slide are stained with cytokeratin-7 and cytokeratin-17, which are both specific to cells of trophoblast origin. Such identifies the cells as trophoblasts which are then easily analyzed using FISH technology. However, other types of genetic screening, analysis, and tests may also be performed on the trophoblasts isolated in the manner set forth herein.

The enriching media and methodologies taught herein promote preservation and viability of fetal cells immediately after cervical mucus sample collection and during transport to a clinical laboratory, and the chemical reagents employed in the described sequences of processing at the clinical laboratory together result in obtaining an enhanced number of purified, isolated fetal cells from a single sample. This proves to be a significant advance in providing a high quality, high yielding source of trophoblast cells in a condition suitable for FISH or molecular analysis.

A better understanding of the present embodiments and of many advantages should be apparent from the following example, which is to be construed as illustrative and in no way limiting.

### EXAMPLE

The following basic materials are preferably employed:
Phosphate buffered saline (PBS) with BSA, pH 7.4, (Sigma β-3688); 1M Tris-HCl, pH 7.5, Cellgro (VWR 45001-066); 1M magnesium chloride (Sigma M-1028);; Sodium phosphate dibasic dihydrate (Sigma 71637); Sodium phosphate monobasic dihydrate (Sigma 71505); Pronase protease (50,000 U), (Calbiochem VWR 80601-406); P-Galactosidase (1,500 U), (Roche 0 105 031); N-acetyl-L-cysteine, (Sigma A9165-25 g); DNase I (150,000 U) (Sigma D-5033); Sodium azide (Sigma S-8032); and Hams F-12 Media, HyClone (VWR 16777-488).

Preparation of Specific Reagents.
A. Phosphate Buffer (0.2M phosphate/1.5M NaCl pH 8.0): 7.8 g sodium phosphate monobasic dihydrate, 8.9 sodium phosphate dibasic dihydrate, 43.83 g sodium chloride, and 450 ml sterile water are added to a sterile 500 ml bottle. The mixture is stirred with a magnetic stir bar until completely dissolved. The pH is adjusted to 8.0 with 5M sodium hydroxide, and the volume is adjusted to 500 ml with sterile water. Following filtering through a 0.22 μm filter, the final concentration is 0.2M phosphate and 1.5M NaCl.
B. N-acetyl-L-cysteine (300 mg/ml): 18.0 g N-acetyl-L-cysteine and 50 ml phosphate buffer (0.2M phosphate/1.5M NaCl, pH 8.0) are added to a sterile 100 ml bottle. The mixture is stirred with a magnetic stir bar until completely dissolved. 4.44 g sodium hydroxide is added slowly to the 100 ml bottle. The pH is adjusted to 8.0 with 5M sodium hydroxide, and volume is adjusted to 60 ml with phosphate buffer (0.2M phosphate/1.5M NaCl, pH 8.0). After filtering through a 0.22 μm filter, 6 ml aliquots are dispensed into sterile 15 ml conical tubes.
C. DNase I Storage Buffer: 19.58 ml sterile water, 400 μl 1M Tris-HCl, and 20 μl 1M MgCl₂ are added to a sterile tube. The mixture is filtered through a 0.22 μm filter into a new sterile tube to provide a final concentration of 20 mM Tris-HCl and 1 mM MgCl₂, which is stored refrigerated (2° to 8° C.).
D. DNase I (100 units/μl): DNase I (150,000 U) is dissolved in 1.5 ml of DNase I Storage Buffer and stored refrigerated (2° to 8° C.).
E. Pronase (2500 units/ml): Pronase (50,000 U) is dissolved in 20 ml sterile water to a final concentration of 2500 units/ml and stored refrigerated (2° to 8° C.).
F. β-Galactosidase: β-Galactosidase (1,500 U) is dissolved in 3 ml sterile water to a final concentration of 0.5 unit/μl and stored refrigerated (2° to 8° C.).
G. Enzyme Mix (sufficient for 80 reactions): 200 μl DNase (100 units/μl), 1040 μl; β-Galactosidase (0.5 units/μl); and 3200 μl Pronase (2500 units/ml) are added to a sterile 15 ml conical tube. Such is freshly prepared daily on ice, mixed by inversion, and stored on ice until ready to use.
H. 200× Sodium Azide: 200 mg sodium azide is prepared fresh daily by dissolving in 10 ml of sterile water in a sterile 15 ml conical tube.
I. PBS with 1% BSA: PBS is dissolved in 1 liter of sterile water and filtered through a 0.45 μm filter, and BSA is added to a final concentration of 0.01M PBS, 1% BSA. It is stored refrigerated (2° to 8° C.).
J. MEMS Buffer: 100 ml PBS with 1% BSA, 400 ml Hams F-12 media, 10 μl DNase I (100 units/μl), and 2.5 ml sodium azide are added to a sterile 500 ml bottle and mixed by inversion to provide a final concentration of 2 units/ml DNase I and 1× sodium azide, which is stored refrigerated (2° to 8° C.).

### Typical Trophoblast Isolation from Cervical Mucus

All work is performed in a biohazard hood with vacuum hookup. Enzyme mix is prepared as described above and stored on ice until ready to use.

### Processing the Original (First) Set of Sample Tubes

The original (first) set of sample tubes, each containing a collection brush carrying a mucus sample, is incubated in a 37° C. water bath for 30 minutes. These sample tubes are removed from the water bath, and the following reagents are added to each of the sample tubes: 22 μl of N-acetyl-L-cysteine solution (300 mg/ml), pH 8.0, to provide a final concentration of 0.5 mg/ml; 39 μl β-galactosidase solution (0.5 unit/μl) to provide a final concentration of 1.5 units/ml; and 28 μl of 1 M CaCl₂. The reagents are mixed by placing the tubes on a tube rocker in a 37° C. incubator for 30 minutes. The first set of sample tubes is then retrieved from the incubator, and 55.5 μl of chilled enzyme mix is added to each of the tubes. The tubes are again placed on a tube rocker in a 37° C. incubator for 2 minutes.

After 2 minutes, the cytobrush is removed from each of the first set of sample tubes with sterile forceps and placed into one of a second set of pre-warmed sample tubes that have been prepared as follows:

a. A set of sterile 15 ml conical tubes are labeled with sample ID numbers.
b. 13 ml Hams F-12 media is aliquoted into each tube.
c. 22 μl N-acetyl-L-cysteine solution (300 mg/ml), pH 8.0, is added to each tube to a final concentration of 0.5 mg/ml.
d. 39 μl β-galactosidase solution (0.5 unit/μl) is added to each tube to a final concentration of 1.5 units/ml.
e. 28 μl of 1 M CaCl₂ is added to each tube.
f. The tubes are incubated in a 37° C. water bath for at least 10 minutes.

The second set of sample tubes now containing the cytobrushes are concurrently processed with the processing of the supernatant from the first set of sample tubes.

### Processing the Supernatant from the First Set of Sample Tubes

The first set of sample tubes containing the supernatant following removal of the cytobrushes is placed on tube rocker in a 37° C. incubator for 10.5 minutes. Next, 13 μl of 0.5 M EDTA is added to each sample tube, and the sample tubes are centrifuged at 500 g (1466 rpm) for 5 minutes at 37° C. Supernatant is then vacuum-aspirated out of each tube to leave a volume of about 500 μl, including the pellet, in media, and the volume is then adjusted to approximately 13 ml with Hams F-12 media at 37° C. The tubes are centrifuged at 500 g (1466 rpm) for 5 minutes at 37° C. Supernatant is again vacuum-aspirated out of each tube to leave a volume of about 500 μl, including a pellet, in media, and the volume in each sample tube is again adjusted to approximately 13 ml with Hams F-12 media at 37° C. The tubes are again centrifuged at 500 g (1466 rpm) for 5 minutes at 37° C. Supernatant is vacuum-aspirated out of each tube to leave a volume of about 500 μl, including a pellet, in media. The cells are resuspended in 5 ml of microchannel buffer at 37° C., and the tubes are centrifuged at 500 g (1466 rpm) for 5 minutes at 37° C. Supernatant is vacuum-aspirated out of each of the first set of sample tubes, leaving the washed cells in a volume of about 300 μl channel buffer.

### Processing the Second Set of Sample Tubes

The second set of sample tubes containing cytobrushes is placed on a tube rocker in a 37° C. incubator for 15 minutes. Then, 55.5 μl of the chilled enzyme mix is added to each tube, and the tubes are placed on a tube rocker in a 37° C. incubator for 3 minutes.

The cytobrush is then removed with sterile forceps from each of the second set of sample tubes, and it is placed into one of a third set of pre-warmed sample tubes, prepared as follows:
a. A set of sterile 15 ml conical tubes are labeled with sample ID numbers.
b. 13 ml Hams F-12 media is aliquoted into each tube.
c. 22 μl N-acetyl-L-cysteine solution (300 mg/ml), pH 8.0, is added to each tube to a final concentration of 0.5 mg/ml.
d. 39 μl β-galactosidase solution (0.5 unit/μl) is added to each tube to a final concentration of 1.5 units/ml.
e. 28 μl of 1 M CaCl₂ is added to each tube.
f. The tubes are incubated in a 37° C. water bath for at least 10 minutes.

The third set of sample tubes containing the cytobrushes are concurrently processed with the processing of the supernatant from the second set of sample tubes.

### Processing the Supematant from the Second Set of Sample Tubes

The second set of sample tubes containing the supernatant following removal of the cytobrushes is placed on tube rocker in a 37° C. incubator for 9.5 minutes. Next, 13 μl of 0.5 M EDTA is added to each sample tube, and the sample tubes are centrifuged at 500 g (1466 rpm) for 5 minutes at 37° C. Supernatant is then vacuum-aspirated out of each tube to leave a volume of about 500 μl, including the pellet, in media, and the volume is then adjusted to approximately 13 ml with Hams F-12 media at 37° C. The tubes are centrifuged at 500 g (1466 rpm) for 5 minutes at 37° C. Supernatant is again vacuum-aspirated out of each tube to leave a volume of about 500 μl, including a pellet, in media, and the volume in each sample tube is again adjusted to approximately 13 ml with Hams F-12 media at 37° C. The tubes are centrifuged at 500 g (1466 rpm) for 5 minutes at 37° C., and supernatant is vacuum-aspirated out of each tube to leave a volume of about 500 μl, including a pellet, in media. The cells are resuspended in 5 ml of microchannel buffer at 37° C., and the tubes are centrifuged at 500 g (1466 rpm) for 5 minutes at 37° C. Supernatant is vacuum-aspirated out of each of the second set of sample tubes to leave the washed cells in a volume of about 300 μl microchannel buffer.

### Processing the Third Set of Sample Tubes

The third set of sample tubes containing cytobrushes is placed on a tube rocker in a 37° C. incubator for 15 minutes. Then, 55.5 μl of the chilled enzyme mix is added to each tube, and the tubes are placed on a tube rocker in a 37° C. incubator for 5 minutes. The cytobrush is removed from each of the third set of sample tubes with sterile forceps and placed into a sterile 15 ml tube labeled with the sample ID number. The removed cytobrushes are stored at 4° C.

The third set of sample tubes containing the supernatant following removal of the cytobrushes is placed on tube rocker in a 37° C. incubator for 9.5 minutes. Next, 13 μl of 0.5 M EDTA is added to each sample tube, and the sample tubes are centrifuged at 500 g (1466 rpm) for 5 minutes at 37° C. Supernatant is then vacuum-aspirated out of each tube to leave a volume of about 500 μl, including the pellet, in media. The volume is then adjusted to approximately 13 ml with Hams F-12 media at 37° C., and the tubes are again centrifuged at 500 g (1466 rpm) for 5 minutes at 37° C. Supernatant is again vacuum-aspirated out of each tube to leave a volume of about 500 μl, including a pellet, in media. The volume in each sample tube is again adjusted to approximately 13 ml with Hams F-12 media at 37° C., and the tubes are again centrifuged at 500 g (1466 rpm) for 5 minutes at 37° C. Supernatant is vacuum-aspirated out of each tube to leave a volume of about 500 μl, including a pellet, in media. The cells are resuspended in 5 ml of channel buffer at 37° C., and the tubes are centrifuged at 500 g (1466 rpm) for 5 minutes at 37° C. Supernatant is vacuum-aspirated out of each sample tube to leave the washed cells in a volume of about 300 μl microchannel buffer.

To isolate the trophoblasts remaining from the sample of cervical mucus, antibodies to Trop-1 and Trop-2 are used. The interior surfaces throughout the collection region in a microchannel device are first derivatized by incubating for 30 minutes at room temperature with a 10 volume % solution of Dow Corning Z-6020. After washing with ethanol, they are treated with nonfat milk at room temperature for about one hour to produce a thin casein coating. Following washing with 10% ethanol in water, they are coated with a hydrogel that is based on isocyanate-capped PEG triols having an average MW of about 6000. A hydrogel prepolymer solution made from 1 part by weight polymer to 6 parts of organic solvent, i.e. acetonitrile (Acn) and DMF, is mixed with a 1 mg/ml antibody solution in 100 mM sodium borate, pH 8.0, containing BSA when coating is ready to begin. The specific coating formulation comprises 100 mg prepolymer in Acn/DMF; 350 μL of 0.25 mg/ml Antibody Mix in aqueous borate buffer; and 350 μL of 1 mg/ml BSA in aqueous borate buffer; the coating formulation contains about 2% polymer by weight. About 5 micrograms total of thiolated anti-Trop-1 and -2 in such aqueous solution, at a concentration of about 0.5 mg/ml, are supplied to a microchannel device, and the solution is left to incubate for 2 hours at 25° C. Following this incubation period, the microchannel device is flushed with a 1% PBS/BSA to provide the antibody-coated surfaces designed for sequestering fetal trophoblast cells.

It may be desirable to use 3 such microflow separation devices in parallel, and if so, about 300 μl is passed through each Trop-1 and Trop-2 coated microchannel device by connecting the device outlet tubing to a vacuum pump and supplying this cell suspension to a vertically oriented inlet. The pump is operated to produce a slow continuous flow of the sample liquid through the device at room temperature, preferably at a rate of about 3-5 μl/min. During this period, the Trop-1 and Trop-2 Abs capture trophoblasts that are present in the sample. After the entire sample is delivered to each, a slow flushing is carried out with a 1% PBS/BSA aqueous buffer. About 100 μl of this aqueous buffer is fed through the device over a period of about 10 minutes, which removes all non-specifically bound biomaterial from the flow channel in the device. Two additional washings are carried out, each with about 100 μl of 1% PBS plus 1% BSA, over periods of about 10 minutes to assure complete removal.

Following the completion of washing, the interior of the device is flooded with a 0.25% solution of trypsin, and the inlet and outlet to and from the device are blocked with stoppers. The device is incubated in a horizontal orientation for about 20 minutes at 27° C. At the completion of this time period, the device is loaded into a centrifuge and spun at 500 g for about 5 minutes, causing the now-detached cells to be forced by centrifugal force against the surface of the hydrogel-coated flat slide. At the end of centrifuging, the aqueous trypsin solution is drained from the device, and the device is dried. The body of the device is carefully separated from the underlying flat slide. The cells adhering to the surface of the slide are stained with cystokeratin-7 and cytokeratin-17, which are specific to cells of trophoblast origin, thus identifying the trophoblast cells which are then easily analyzed using FISH technology.

Although the invention has been described with regard to certain preferred embodiments which constitute the best mode presently known to the inventor for carrying out this invention, it should be understood that various changes and modifications as would be obvious to one having ordinary skill in this art may be made without departing from the scope of the invention which is defined in the claims which follow. For example, although certain preferred reagents for use in the purification of the sample are described, other materials may be employed as are well known in this art as being suitable for these purposes. Although the emphasis has generally been upon the separation of fetal trophoblasts from a cervical mucus extract by sequestering the trophoblasts, it should be understood that such a sample could be treated by negative enrichment to target a group of unwanted cells which would then be captured.

The disclosures of all US patents and applications specifically identified herein are expressly incorporated herein by reference. Particular features of the invention are emphasized in the claims which follow.

1-15. (canceled)

16. A method for isolating fetal trophoblast cells from cervical mucus compnsing:
(a) incubating a mixture containing a sample obtained from cervical mucus with a nuclease and a protease; and
(b) isolating fetal trophoblast cells from the mixture.

17. The method of claim 16 further comprising incubating the mixture with a mucolytic agent and a sugar hydrolysis enzyme prior to incubating the mixture with the nuclease and the protease.

18. The method of claim 16, wherein the mixture is incubated at a temperature of between about 35° C. and about 40° C.

19. The method of claim 16, wherein the mixture is incubated at about 37° C.

20. The method of any one of claims 18 or 19 wherein the mixture is incubated for between about 2 minutes and about 5 minutes.

21. The method of claim 17, wherein the mixture is incubated with the mucolytic agent and the sugar hydrolysis enzyme at a temperature of between about 35° C. and about 40° C.

22. The method of claim 17, wherein the mixture is incubated with the mucolytic agent and the sugar hydrolysis enzyme at a temperature of about 37° C.

23. The method of any one of claims 21 or 22, wherein mixture is incubated with the nuclease and the protease for between about 10 minutes and about 30 minutes.

24. The method of claim 16, wherein the nuclease is an endonuclease, an exonuclease, a restriction enzyme, a DNase, DNase I, mung bean nuclease or Benzonase®.

25. The method of claim 16, wherein the protease is dispase, pronase, trypsin, chymotrypsin, pepsin or papain.

26. The method of claim 16, wherein the nuclease is DNase I and the protease is pronase.

27. The method of claim 17, wherein the mucolytic agent is N-acetyl cysteine, dithiothreitol, bromhexine hydrochloride, L-cysteine, hyaluronate lyase, hyaluronoglucosaminidase, hyaluronoglucosaminidase or a hyaluronidase.

28. The method of claim 17, wherein the mucolytic agent is N-acetyl cysteine and the sugar hydrolysis enzyme is β-galactosidase.

29. The method of claim 17, wherein the nuclease is DNase I, the protease is pronase, the mucolytic agent is N-acetyl cysteine and the sugar hydrolysis enzyme is β-galactosidase.

30. The method of claim 16, further comprising contacting the mixture with EDTA, ECTA or a detachment enzyme.

31. The method of claim 30 further comprising:
(a) centrifugating the mixture to concentrate the cells
(b) resuspending the cells;
(c) centrifugating the cells;
(d) resuspending the cells; and
(e) centrifugating the cells.

32. The method of claim 16, further comprising isolating fetal trophoblast cells from maternal cells with a binding entity on a solid surface, wherein the binding entity specifically binds fetal trophoblast cells.

33. The method of claim 32, wherein the binding entity is on a surface of a microchannel device.

34. The method of claim 32, wherein the binding entity is an antibody.

35. A kit for isolating fetal trophoblast cells from cervical mucus comprising:
a nuclease; and
a protease; and
a set of written instruction describing the use of the kit to isolate fetal trophoblast cells from cervical mucus.

36. The kit of claim 35 further comprising:
a mucolytic agent; and
a sugar hydrolysis enzyme.

37. The kit of claim 35 or claim 36 further comprising:
a binding entity;
wherein the binding entity specifically binds fetal trophoblast cells.

38. A method for isolating fetal trophoblast cells from cervical mucus comprising:
(a) depositing a sample obtained from cervical mucus in a media that selectively preserves fetal trophoblast cells over maternal cells;
(b) removing the sample from the media;
(c) incubating a mixture containing the sample obtained from cervical mucus with a mucolytic agent and a sugar hydrolysis enzyme at a temperature between about 35° C. and about 40° C. for between about 10 and about 15 minutes;
(d) incubating the mixture with a nuclease and a protease at a temperature between about 35° C. and about 40° C. for between about 2 and about 5 minutes.
(e) contacting the mixture with EDTA;
(f) centrifugating the mixture to concentrate the cells; and
(g) resuspending the cells in a media formulated to grow CHO cells.

39. The method of claim 38 further comprising:
(a) centrifugating the cells;
(b) resuspending the cells in an aqueous buffer containing azide; and
(c) isolating fetal trophoblast cells from maternal cells with antibodies on a surface of a microchannel device wherein the antibodies specifically binds fetal trophoblast cells.
